(19) United States
(12) Patent Application Publication
(10) Pub. No.: US 2026/0263824 A1
(43) Pub. Date: Sep. 10, 2026

(54) Medical System Comprising an Implantable Medical Device and Pressure Sensor

(71) Applicant: BIOTRONIK SE & Co. KG, Berlin (DE)

(72) Inventors: Dominic WIST, Berlin (DE); Bjoern DIEM, Berlin (DE); Thomas KRUCZYK, Berlin (DE)

(73) Assignee: BIOTRONIK SE & Co. KG, Berlin (DE)

(21) Appl. No.: 19/166,464

(22) PCT Filed: Mar. 5, 2024
(86) PCT No.: PCT/EP2024/055673

§ 371 Date: Sep. 17, 2025

(30) Foreign Application Priority Data
Mar. 22, 2023 (EP) 23163433.8

(51) Int. Cl. A61N 1/39

(52) U.S. Cl. CPC A61N 1/3987; A61N 1/3956

(57) ABSTRACT

A medical system including an implantable medical device for applying defibrillation shock pulses to a patient's heart to treat an acute cardiac disorder. The medical system includes an implantable pressure sensor for measuring the patient's blood pressure, wherein the medical device and the pressure sensor are configured to communicate with each other. The medical device is configured to decide whether a shock is applied after communicating with the pressure sensor.

### CROSS-REFERENCE TO RELATED APPLICATIONS

This application is the United States National Phase under 35 U.S.C. § 371 of PCT International Patent Application No. PCT/EP 2024/055673, filed on Mar. 5, 2024, which claims the benefit of European Patent Application No. 23163433.8, filed on Mar. 22, 2023, the disclosures of which are hereby incorporated by reference herein in their entireties.

### TECHNICAL FIELD

The instant invention concerns a medical system, a method for operating the system, a method for setting up the system, an implantable medical device for applying defibrillation shock pulses, and an implantable pressure sensor for measuring a patient's blood pressure.

### BACKGROUND

A medical system generally comprises an implantable medical device for applying defibrillation shock pulses to a patient's heart. Through the shock pulses, an acute cardiac disorder may be treated. Such a medical device may be an implantable cardioverter defibrillator (ICD), in particular, a subcutaneous implantable cardioverter defibrillator (S-ICD). It is known that approximately 30% of the shocks delivered by these medical devices are inappropriate. That is, the medical device mistakenly recognizes a need to apply the shock pulse in the absence of an acute cardiac disorder. Inappropriate shocks can cause pain and severe anxiety in the patient.

Typically, the medical device comprises a sensing arrangement comprising at least one electronic lead for sensing an electrocardiogram signal. The detection of the cardiac disorder is based on the electrocardiogram signal. If the electronic lead is subject to electromagnetic radiation or electromagnetic interference (EMR, EMI) or suffers from a lead fracture, it may detect misleading signals, for example, detect an electrocardiogram signal that is similar to an arrhythmic signal in the absence of an arrhythmia. Based on an evaluation of such a signal, the medical device may decide to apply a painful shock pulse despite the lack of a true medical indication to do so.

Prevention of unnecessary shocks due to lead fracture may be achieved by regular checks of the implanted medical device through a physician. These checks include long-term monitoring of a lead impedance trend, whereby the lead impedance is known to slowly decrease over time. An unusual increase in lead impedance against the trend may signal a beginning fraction of a lead which may, in turn, increasingly cause inappropriate shock delivery.

In case of a supraventricular tachycardia (SVT) it is known to use an SVT discrimination algorithm to avoid unnecessary shocks. Another option is an antitachycardia pacing (ATP) algorithm, in particular in an ICD, that might avoid shock delivery. These algorithms may operate on a signal from the at least one electronic lead of the medical device so that they are, in principle, also prone to lead fracture.

In another scenario, an arrhythmia may be correctly detected. The arrhythmia, however, does not destabilize the hemodynamics so that there is actually no acute need to apply a shock pulse. The medical device will apply a shock pulse, nonetheless, based on the electrocardiogram signal so that the patient, who is most likely conscious under such hemodynamically stable conditions, suffers from the needlessly inflicted pain.

Some cardiac disorders only persist for a limited amount of time, for example in case of a ventricular tachycardia or ventricular fibrillation episode. In such cases, unnecessary shocks are usually avoided by the following steps: The medical device may, firstly, detect the disorder, then prepare to apply the shock pulse and, lastly, check whether the disorder still persists. The preparation for applying the shock pulse may comprise charging a capacitor of the medical device with an appropriate amount of energy. Triggering the release of the energy is made dependent on the result of the last check whether the disorder persists. If the disorder has vanished, the release is not triggered because it is assumed that the patient is in an uncritical, stable situation and the cause for the shock delivery does not exist anymore. Making shock delivery dependent on the last check of whether the disorder persists, is sometimes called “last look principle”.

Taking the above-described challenges into consideration, the patient's well-being would benefit if the number of inappropriately delivered shock pulses through the medical device were reduced.

The present disclosure is directed toward overcoming one or more of the above-mentioned problems, though not necessarily limited to embodiments that do.

### SUMMARY

It is, therefore, an object of the present invention to provide a medical system comprising an implantable medical device for applying defibrillation shock pulses to a patient's heart to treat an acute cardiac disorder that avoids unnecessary shock pulses.

According to a first aspect of the invention, this object is achieved by providing a medical system according to claim 1.

Accordingly, the medical system comprises an implantable pressure sensor for measuring the patient's blood pressure, wherein the medical device and the pressure sensor are configured to communicate with each other. The medical device is configured to decide whether a shock is applied after communicating with the pressure sensor.

The implantable pressure sensor may be a pulmonary artery pressure (PAP) monitor. It may be implanted at the pulmonary artery. Such a location makes it desirable to design the pressure sensor as small as possible, e.g., provide it with a small battery, so that it does not block the blood flow and to reduce the need for maintenance and repair of the pressure sensor, e.g., exchange of the battery, to an absolute minimum. Thus, the pressure sensor may be in a sleep state by default in order to save battery life.

The pressure sensor may be configured to provide a second set of data in addition to a first set of data measured by the medical device to characterize an acute cardiac disorder. The first set of data may include the electrocardiogram signal detected by the sensing arrangement including at least one electrode lead of the medical device. The second set of data may include any information on blood pressure, for example its cyclic variations or a pulse.

The communication between the medical device and the pressure sensor may include transmitting and receiving information from each other. By communicating with each other, the medical device and pressure sensor may decrease the risk of the medical device applying unnecessary shock pulses.

The medical device may apply two types of shocks: a first shock and post shocks after the first shock which have a lower energy than the first shock. Unnecessary shock pulses may be avoided for both types of shocks.

According to the invention, the medical device is configured to decide whether a shock is applied after communicating with pressure sensor. The decision whether to apply a shock may be preceded by a detection of an acute cardiac disorder by the medical device that requires treatment and, optionally, by charging of a capacitor of the medical device so that the shock is ready to be delivered immediately with the decision. By deciding whether the shock is applied only after communicating with the pressure sensor, the medical device may ensure that the shock is actually necessary.

According to an exemplary embodiment, a high-energy shock (first shock) may deliver a large amount of electric energy into the target area. The energy may remain after delivery of the shock and disturb the electrocardiogram signal so that this signal cannot necessarily be relied on—the medical device may be said to be temporarily “blind” to an intrinsic rhythm of the heart. Such a situation may bear a risk to reinitiate the arrhythmia when pacing “blindly” or harm the patient's well-being. Pacing in an SICD (or any other suitable medical device) means delivery of low-energy shocks. Thus, it may be particularly useful to make the decision whether to apply pacing re-initiation shocks after a defibrillation to restore the natural pacemaker of the heart after communicating with pressure sensor since the pressure sensor may not be impacted by the remains of the shock energy.

In one embodiment, the medical device and the pressure sensor are configured to communicate with each other wirelessly, in particular, through intra-body communication. A wireless communication between the medical device and the pressure sensor may be established through e.g. Bluetooth, Bluetooth Low Energy, MICS band, MEDS band, ISM band, low energy coil telemetry, galvanic coupled telemetry.

In one embodiment, the medical device is configured to transmit a wake-up signal to the pressure sensor to wake up the pressure sensor from a sleep state so that the pressure sensor can measure the blood pressure. The pressure sensor may be operated in at least two states. One of the at least two states may be the sleep state. In the sleep state the pressure sensor may be configured not to measure the blood pressure so that battery life is saved. It may be switched off in the sleep state. Another one of the at least two states may be an operational state in which the pressure sensor may be configured to (continuously) measure the blood pressure.

In principle, the medical device may be configured to transmit a command signal to the pressure sensor to switch the pressure sensor from the sleep state to the operational state and vice versa. The wake-up signal as a form of such a command signal may switch the pressure sensor from the sleep state to the operational state.

In a further embodiment, the wake-up signal comprises an RF signal and/or a magnetic pulse. The RF signal may be a high-energy RF signal, e.g., an RF signal from an energy range in the upper half of the RF spectrum, in particular, in the range of 3 to 30 MHz. The signal may allow the medical device to transmit a sufficient amount of energy to the pressure sensor so that it is ensured that the pressure sensor wakes up when receiving the signal.

According to an embodiment, the medical device is configured to send a near-zero-power wake-up signal to the pressure sensor. The near-zero power wake-up signal is generated e.g. by a specific signal circuit and/or via specific sensors. See for instance the technology cited in “A Near-Zero-Power Wake-Up Receiver Achieving −69-dBm Sensitivity”, IEEE JOURNAL OF SOLID-STATE CIRCUITS, 0018-9200, 2018 IEEE; “Broadband Zero-Power Wakeup MEMS Device for Energy-Efficient Sensor Nodes”, Micromachines 2022, 13(3), 407; “Near-Zero Power Wake-Up Receivers for the Internet of Things” Sep. 27, 2019, Moody, Jesse, Electrical Engineering—School of Engineering and Applied Science, University of Virginia.

Moreover, according to an embodiment of the present invention, the medical device is configured to send wake-up signal to the pressure sensor which is based on intra body communication, in particular galvanic coupled telemetry. The galvanic coupled telemetry is performed by a first device (medical device) which comprises means to change an electrical field of the body, and a second device (pressure sensor) which is able to detect the changes of the electrical field of the same body. The changes of the electrical field are modulated in a way by the first device that a specific signal or pattern can be recognized by the second device, which are interpreted as commands, e.g. a wake-up. According to an embodiment, the first and the second device are configured to perform bidirectional communication based on galvanic coupled telemetry.

In one embodiment, the pressure sensor is further configured to transmit to the medical device data regarding the blood pressure and/or a shock prevention signal wherein the medical device is configured to decide whether or not to apply a shock based on the information and/or the shock prevention signal. When the pressure sensor is in the operational state and measures the blood pressure, it may obtain information about the blood pressure. This information may be forwarded to the medical device. By receiving the information, the medical device may obtain the second set of data in addition to the first set of data measured by the medical device itself so that it may more reliably decide whether or not to apply the shock and, thus, avoid unnecessary shocks. The medical device may, for example, be configured to assess the hemodynamic relevance of the arrhythmia based on the two sets of data. A painful shock may be delayed if it finds that the arrhythmia is hemodynamically stable. The delay may be used to perform prolonged ATP algorithm trials.

Alternatively or additionally, the pressure sensor may be configured to carry out an (at least basic) analysis of the information so that it is able to decide whether or not to send a shock prevention signal.

The analysis at the pressure sensor (and, alternatively, an analysis at the medical device preceding the decision to apply the shock) may comprise determining if a cyclic variation of the blood pressure is within a predetermined range, for example, within a range of a heart rate, e.g., a duration below 5 s, below 1 s or below 600 ms (for one cycle). Alternatively, it may be determined whether the cyclic variations are within a predetermined range with respect to their maxima, e.g., in a range of 17 to 32 mm Hg (the systolic pressure in the pulmonary artery if the pressure sensor is located there), and/or with respect to their minima, e.g., 4 to 13 mm Hg (the diastolic pressure in the pulmonary artery if the pressure sensor is located there). It is also conceivable, to characterize the cyclic variations by a mean pressure, which may be 9 to 19 mm Hg in the pulmonary artery. Hence, the cyclic variations may be characterized by a predetermined range of their amplitude additionally or alternatively to the characterization by their duration. If the cyclic variation is out of the predetermined range (or no cyclic variation is detected at all), the shock prevention signal may be transmitted. By having the pressure sensor only send the shock prevention signal, the data to be transmitted may be minimized so that reliable and fast reception at the medical device side is more likely.

According to embodiments of the present invention, the pressure sensor is placed in the pulmonary artery. In general, the present invention is applicable to other forms of blood pressure sensors and other ways of blood pressure sensing as well.

The medical system may be designed such that if the medical device does not receive any information from the pressure sensor, a shock pulse will be applied. Thereby, a safeguard may be realized by which the patient's health is protected in any case even if there is a risk that the well-being is harmed due to unnecessarily applied shocks. Thus, in one embodiment, the medical device is configured to wait for a predetermined amount of waiting time after sending the wake-up signal and to apply a shock if no communication from the pressure sensor is received after the waiting time has passed. The waiting time may give the pressure sensor sufficient time to wake up, perform a measurement and communicate with the medical device, e.g., by sending a shock prevention signal-these steps may take a few seconds, e.g. 1 to 5 s which corresponds to the waiting time. In case the pressure sensor is unable to communicate with the medical device (e.g., due to low battery), the medical device will proceed to apply the shock pulse.

In one embodiment, the information (transmitted from the pressure sensor to the medical device) comprises information on the presence or absence of cyclic variations of the blood pressure within a predetermined range and/or information on a detected blood pulse. If the information is provided to the medical device, the medical device may carry out an analysis of the information so that it is able to base its decision whether or not to apply a shock pulse on a more profound analysis of the situation.

In the presence of cyclic variations, it may not be necessary to apply a shock pulse immediately (application of the shock pulse may be postponed) if the cyclic variations are within a predetermined range as mentioned above. It may also not be necessary to deliver post-shock pacing in the presence of such cyclic variations. In the absence of cyclic variations, however, a shock pulse or a post-shock pulse may be applied. Taking the absence of cyclic variations into consideration may be particularly useful in case of a ventricular fibrillation, wherein the medical device may detect an electric signal (and potentially conclude that the hemodynamics are stable) but the absence of the cyclic variations in the blood pressure may reveal that pacing is required. Application of a shock pulse may also be necessary in case the cyclic variations are beyond a predetermined range. This may, for example, be the case if the cyclic variations of the blood pressure are merely due to respiration. In principle, the decision at the pressure sensor whether or not to send a shock prevention signal may be based on a similar analysis of the information.

In the presence of a blood pulse, it may also not be necessary to apply a shock pulse, in particular, if the blood pulse is within a predetermined frequency window. If the information on the detected blood pulse reveals, that there is no blood pulse or that the blood pulse is outside the predetermined frequency window, the medical device may decide to apply a shock pulse.

In one embodiment, the pressure sensor is configured to transmit the information on the detected blood pulse in the form of a plurality of individual signals, wherein each of the plurality of signals represents a single detected pulse beat. The individual signals may be transmitted as one for each pulse beat and once the pulse beat is detected. Such a configuration of the pressure sensor may allow the medical device to obtain the information on the detected blood pulse in real time so that it may decide to apply a shock pulse once it is sufficiently certain that application of the shock pulse is not inappropriate. The pressure sensor may further be configured to transmit the individual signals for a predetermined amount of measuring time and to switch back to sleep mode after the predetermined amount of measuring time has passed so that the battery life of the pressure sensor is conserved.

In order to achieve a sufficient certainty whether or not to apply the shock pulse, the medical device may compare the pulse beat with an electrical signal measured by the medical device itself to determine whether the electrical signal represents real heart beats. Thus, in one embodiment, the medical device comprises a sensing arrangement for sensing an electrocardiogram signal, wherein the medical device is configured to compare the detected blood pulse with the electrocardiogram signal to cross-check the presence of heart beats. By cross-checking the presence of heart beats through a first set of data relating to an electrocardiogram signal and a second set of data including information on a pulse (or, generally, information on the blood pressure), the number of unnecessarily applied shock pulses may be significantly reduced.

According to a second aspect of the invention, the object is also achieved by providing a method for operating the system of the first aspect of the invention comprising the steps of: determining, by the medical device, that a shock is required; transmitting, by the medical device, a wake-up signal to a pressure sensor to wake up the pressure sensor from a sleep state; detecting, by the pressure sensor, the blood pressure; transmitting, by the pressure sensor, to the medical device nothing or transmitting information regarding the blood pressure and/or a shock prevention signal; and deciding, by the medical device, whether or not a shock is applied.

In one embodiment, deciding, by the medical device, whether or not a shock is applied comprises delaying application of the shock by a delay time if it decides not to apply a shock and determining, again, that a shock is required after the delay time has passed. In principle, when the medical device decides not to apply a shock immediately, a shock may be necessary at a later point in time (e.g., a few seconds to one minute later), unless the medical device detected in the meantime that no shock is required anymore. It is therefore advantageous to postpone the application (instead of cancelling it). During the delay time, the medical device may actively monitor an electrical signal of the heart to determine whether or not a shock is required. In particular, a capacitor of the medical device may be kept charged with an appropriate amount of electrical energy (and not be discharged).

According to a third aspect of the invention, the object is achieved by providing a method for setting up the system according to the first aspect of the invention comprising the steps of: providing a programming device configured to communicate with the medical device and the pressure sensor; informing, by the programming device, the medical device of the presence of the pressure sensor and the pressure sensor of the presence of the medical device; and communicating, by the medical device, with the pressure sensor.

Such a method may be used during an interrogation process. A cardiologist may use the programming device during the interrogation process to communicate with the medical system. The programming device is configured to detect the presence of the medical device and the pressure sensor. The medical device and the pressure sensor may each store a set of parameters which may be interrogated by the programming device. The set of parameters may comprise an indication of whether or not each of the devices knows of the presence of the other one. Through obtaining the set of parameters, the programming device may be able to check whether the medical device knows of the pressure sensor and the pressure sensor knows of the medical device. The interrogation process may be carried out whilst the medical device and pressure sensor are implanted with the patient.

The instant invention concerns a medical system, a method for operating the system, a method for setting up the system, an implantable medical device for applying defibrillation shock pulses, and an implantable pressure sensor for measuring a patient's blood pressure.

A medical system generally comprises an implantable medical device for applying defibrillation shock pulses to a patient's heart. Through the shock pulses an acute cardiac disorder may be treated. Such a medical device may be an implantable cardioverter defibrillator (ICD), in particular, a subcutaneous implantable cardioverter defibrillator (S-ICD). It is known that approximately 30% of the shocks delivered by these medical devices are inappropriate. That is, the medical device mistakenly recognizes a need to apply the shock pulse in the absence of an acute cardiac disorder. This can cause pain and severe anxiety in the patient.

Typically, the medical device comprises a sensing arrangement comprising at least one electronic lead for sensing an electrocardiogram signal. The detection of the cardiac disorder is based on the electrocardiogram signal. If the electronic lead is subject to EMR or suffers from a lead fracture, it may detect misleading signals, for example, detect an electrocardiogram signal that is similar to an arrhythmic signal in the absence of an arrhythmia. Based on an evaluation of such a signal, the medical device may decide to apply a painful shock pulse despite the lack of a true medical indication to do so.

Prevention of unnecessary shocks due to lead fracture may be achieved by regular checks of the implanted medical device through a physician. These checks include long-term monitoring of a lead impedance trend whereby the lead impedance is known to slowly decrease over time. An unusual increase in lead impedance against the trend may signal a beginning fraction of a lead which may be a course of inappropriate shock delivery.

In case of a supraventricular tachycardia (SVT) it is known to use an SVT discrimination algorithm to avoid unnecessary shocks. Another option is an antitachycardia pacing (ATP) algorithm, in particular in ICD, that might avoid shock delivery. These algorithms may operate on a signal from the at least one electronic lead of the medical device so that they are, in principle, also prone to lead fracture.

In another scenario, an arrhythmia may be correctly detected. The arrhythmia, however, does not destabilize the hemodynamics so that there is actually no acute need to apply a shock pulse. The medical device will apply a shock pulse nonetheless based on the electrocardiogram signal so that the patient, who is most likely conscious under these conditions, suffers from the needlessly inflicted pain.

Some cardiac disorders only persist for a limited amount of time, for example in case of a ventricular tachycardia or ventricular fibrillation episode. In such a case, unnecessary shocks are usually avoided by the following steps: The medical device may, firstly, detect the disorder, then prepare to apply the shock pulse and, lastly, check whether the disorder still persists. The preparation for applying the shock pulse may comprise charging a capacitor of the medical device with an appropriate amount of energy. Triggering the release of the energy is made dependent on the result of the last check whether the disorder still persists. If the disorder has vanished, the release is not triggered because it is assumed that the patient is in an uncritical, stable situation and the cause for the shock delivery does not exist anymore. Making shock delivery dependent on the last check of whether the disorder still persists, is sometimes called “last look principle”.

Taking the above-described challenges into consideration, the patient's well-being would benefit if the number of inappropriately delivered shock pulses through the medical device were reduced.

It is, therefore, an object of the present invention to provide a medical system comprising an implantable medical device for applying defibrillation shock pulses to a patient's heart to treat an acute cardiac disorder that avoids unnecessary shock pulses.

According to a first aspect of the invention, this object is achieved by providing a medical system according to claim 1.

Accordingly, the medical system comprises an implantable pressure sensor for measuring the patient's blood pressure, wherein the medical device and the pressure sensor are configured to communicate with each other.

The implantable pressure sensor may be a pulmonary artery pressure (PAP) monitor. It may be implanted at the pulmonary artery. Such a location makes is desirable to design the pressure sensor as small as possible, e.g., provide it with a small battery, so that it does not have a negative hemodynamic impact and to reduce the need for maintenance and repair of the pressure sensor, e.g., exchange of the battery, to an absolute minimum. Thus, the pressure sensor may be in a sleep state by default in order to save battery life.

The pressure sensor may be configured to provide a second set of data in addition to a first set of data measured by the medical device to characterize an acute cardiac disorder. The first set of data may include the electrocardiogram signal detected by the sensing arrangement including at least one electrode lead of the medical device. The second set of data may include any information on blood pressure, for example its cyclic variations or a pulse.

The communication between the medical device and the pressure sensor may include transmitting and receiving information from each other. By communicating with each other, the medical device and pressure sensor may decrease the risk of the medical device applying unnecessary shock pulses.

The medical device may apply two types of shocks: a first shock and post shocks subsequent to the first shock which have a lower energy than the first shock. Unnecessary shock pulses may be avoided for both types of shocks.

In an embodiment, the medical device is configured to decide whether a shock is applied after communicating with pressure sensor. The decision whether to apply a shock may be preceded by a detection of an acute cardiac disorder by the medical device that requires treatment and, optionally, by charging of a capacitor of the medical device so that the shock is ready to be delivered immediately with the decision. By deciding whether the shock is applied only after communicating with the pressure sensor, the medical device may ensure that the shock is actually necessary.

In particular, a high-energy shock (first shock) may deliver a large amount of electric energy into the target area. The energy may remain after delivery of the shock and negatively impacts the electrocardiogram signal so that this signal cannot necessarily be relied on-the medical device may be said to be temporarily “blind” to an intrinsic rhythm of the heart. Such a situation may bear a risk to reinitiate the arrhythmia when pacing “blindly” or harm the patient's well-being since pacing in, for example, an (S-)ICD means delivering of low-energy shocks. Thus, it may be particularly useful to make the decision whether to apply post shocks to restore the natural pacemaker of the heart after communicating with pressure sensor since the pressure sensor may not be impacted by the remains of the shock energy.

In one embodiment, the medical device and the pressure sensor are configured to communicate with each other wirelessly, in particular, through intra-body communication. A wireless communication between the medical device and the pressure sensor may be established through Bluetooth as an example.

In one embodiment, the medical device is configured to transmit a wake-up signal to the pressure sensor to wake up the pressure sensor from a sleep state so that the pressure sensor can measure the blood pressure. The pressure sensor may be operated in at least two states. One of the at least two states may be the sleep state. In the sleep state the pressure sensor may be configured not to measure the blood pressure so that battery life is saved. It may simply be switched off in the sleep state. Another one of the at least two states may be an operational state in which the pressure sensor may be configured to (continuously) measure the blood pressure.

In principle, the medical device may be configured to transmit a command signal to the pressure sensor to switch the pressure sensor from the sleep state to the operational state and vice versa. The wake-up signal as a form of such a command signal may switch the pressure sensor from the sleep state to the operational state.

In a further embodiment, the wake-up signal comprises an RF signal and/or a magnetic pulse. The RF signal may be a high-energy RF signal—that is an RF signal from an energy range in the upper half of the RF spectrum, in particular, in the range of 3 to 30 MHz. In particular, the signal may allow the medical device to transmit a sufficient amount of energy to the pressure sensor so that it is ensured that the pressure sensor wakes up when receiving the signal.

In one embodiment, the pressure sensor is further configured to transmit to the medical device information regarding the blood pressure and/or a shock prevention signal wherein the medical device is configured to decide whether or not to apply a shock based on the information and/or the shock prevention signal. When the pressure sensor is in the operational state and measures the blood pressure, it may obtain information about the blood pressure. This information may be forwarded to the medical device. By receiving the information, the medical device may obtain the second set of data in addition to the first set of data measured by the medical device itself so that it may more reliably decide whether or not to apply the shock and avoid unnecessary shocks. The medical device may, for example, be configured to assess the hemodynamic relevance of the arrhythmia based on the two sets of data. A painful shock may be delayed if it finds that the arrhythmia is hemodynamically stable. The delay may be used to perform prolonged ATP algorithm trials.

Alternatively or additionally, the pressure sensor may be configured to carry out an (at least basic) analysis of the information so that it is able to decide whether or not to send a shock prevention signal. The analysis may comprise determining if a cyclic variation of the blood pressure is within a predetermined range, for example, within a range of a heart rate, e.g., below 5 s, below 1 s or below 600 ms. If, for example, the cyclic variation is out of the predetermined range (or no cyclic variation is detected at all), the shock prevention signal may be transmitted. By having the pressure sensor only send the shock prevention signal, the data to be transmitted may be minimized so that reliable and fast reception at the medical device side is more likely.

The medical system may be designed such that if the medical device does not receive any information from the pressure sensor, a shock pulse may be applied. Thereby, a safeguard may be achieved so that the patient's health is protected in any case even if there is a risk that the well-being is harmed due to unnecessarily applied shocks. Thus, in one embodiment, the medical device is configured to wait for a predetermined amount of waiting time after sending the wake-up signal and to apply a shock if no communication from the pressure sensor is received after the waiting time has passed. The waiting time may give the pressure sensor sufficient time to wake up, perform a measurement and communicate with the medical device, e.g., by sending a shock prevention signal-these steps may take 1 to 5 s. In case the pressure sensor is unable to communicate with the medical device (e.g., due to low battery), the medical device will proceed to apply the shock pulse.

In one embodiment, the information (transmitted from the pressure sensor to the medical device) comprises information on the presence or absence of cyclic variations of the blood pressure within a predetermined range and/or information on a detected blood pulse. If the information is provided to the medical device, medical device may carry out an analysis of the information so that it is able to base its decision whether or not to apply a shock pulse on a more profound analysis of the situation.

In the presence of cyclic variations, it may not be necessary to apply a shock pulse immediately (application of the shock pulse may be postponed) if the cyclic variations are within a predetermined range as mentioned above. It may also not be necessary to deliver post-shock pacing in the presence of such cyclic variations. In the absence of cyclic variations, however, a shock pulse or a post-shock pulse may be applied. Application of a shock pulse may also be necessary in case the cyclic variations are beyond a predetermined range. This may, for example, be the case if the cyclic variations of the blood pressure and are merely due to respiration. In principle, the decision at the pressure sensor whether or not to send a shock prevention signal may be based on a similar analysis of the information.

In the presence of a blood pulse, it may also not be necessary to apply a shock pulse, in particular, if the blood pulse is within a predetermined frequency window. If the information on the detected blood pulse review, that there is no blood pulse or that the blood pulse is outside the predetermined frequency window, the medical device may decide to apply a shock pulse.

In one embodiment, the pressure sensor is configured to transmit the information on the detected blood pulse in the form of a plurality of individual signals, wherein each of the plurality of signals represents a single detected pulse beat. The individual signals may be send one for each pulse beat and once the pulse beat is detected. Such a configuration of the pressure sensor may allow the medical device to obtain the information on the detected blood pulse in real time so that it may decide to apply a shock pulse once it is sufficiently certain that application of the shock pulse is not inappropriate. The pressure sensor may further be configured to transmit the individual signals for a predetermined amount of measuring time and to switch back to sleep mode after the predetermined amount of measuring time has passed so that battery life of pressure sensor is conserved.

In order to achieve a sufficient certainty whether or not to apply the shock pulse, the medical device may compare the pulse beat with an electrical signal measured by the medical device itself to determine whether the electrical signal represents real heart beats. Thus, in one embodiment, the medical device comprises a sensing arrangement for sensing an electrocardiogram signal, wherein the medical device is configured to compare the detected blood pulse with the electrocardiogram signal to cross-check the presence of heart beats. By cross-checking the presence of heart beats through a first set of data relating to an electrocardiogram signal and a second set of data including information on a pulse, the number of unnecessarily applied shock pulses may be significantly reduced.

According to a second aspect of the mention, the object is achieved by providing a method for operating the system of one the first aspect of the invention comprising the steps of: determining, by the medical device, that a shock is required; transmitting, by the medical device, a wake-up signal to a pressure sensor to wake up the pressure sensor from a sleep state; detecting, by the pressure sensor, the blood pressure; transmitting, by the pressure sensor, to the medical device nothing or transmitting information regarding the blood pressure and/or a shock prevention signal; and deciding, by the medical device, whether or not a shock is applied.

In one embodiment, deciding, by the medical device, whether a shock is applied comprises delaying application of the shock by a delay time if it decides not to apply a shock and determining, again, that a shock is required after the delay time has passed. In principle, when the medical device decides not to apply a shock immediately, a shock may be necessary at a later point in time (e.g., 1 to 5 minutes later). It is therefore advantageous to postpone the application (instead of cancelling it). During the delay time, the medical device may actively monitor an electrical signal of the heart to determine whether or not a shock is required. In particular, a capacitor of the medical device may be kept charged with an appropriate amount of electrical energy (and not be discharged).

According to a third aspect of the invention, the object is achieved by providing a method for setting up the system according to the first aspect of the invention comprising the steps of: providing a programming device configured to communicate with the medical device and the pressure sensor; informing, by the programming device, the medical device of the presence of the pressure sensor and the pressure sensor of the presence of the medical device; and communicating, by the medical device, with the pressure sensor.

Such a method may be used during an interrogation process. The programming device may be used during the interrogation process by a cardiologist. The programming device is configured to detect the presence of the medical device and the pressure sensor. The medical device and the pressure sensor may each store a set of parameters which may be interrogated by the programming device. The set of parameters may comprise an indication of whether or not each of the device knows of the presence of the other one. Through obtaining the set of parameters, the programming device and therefore be able, to check whether the medical device knows of the pressure sensor and the pressure sensor nodes of the medical device. The interrogation process may be carried out whilst the medical device and pressure sensor are implanted with the patient.

If the medical device and/or pressure sensor does not know of the presence of the other one, the programming device may be configured to set the corresponding parameter(s) in the medical device and/or the pressure sensor. This could be done in a programming process, which may be carried out after the interrogation process. After setting the corresponding parameter(s), the medical device may be aware of the presence of the pressure sensor so that it can communicate with the pressure sensor. The communication may, in particular, comprise transmitting the wake-up signal to the pressure sensor to wake up the pressure sensor from the sleep state. Analogously, after setting the corresponding parameter(s), the pressure sensor may be aware of the presence of the medical device so that it can communicate with the medical device. The communication may, in particular, comprise that the pressure sensor is woken up by the medical device. In other words, the medical device may be able to trigger the pressure sensor and the pressure sensor may be able to get triggered by the medical device after both of them are informed about presence of each other.

According to a fourth aspect of the invention, the object is achieved by an implantable medical device for applying defibrillation shock pulses to a patient's heart configured to communicate with a pressure sensor for measuring the patient's blood pressure.

According to a fifth aspect of the invention, the object is achieved by an implantable pressure sensor for measuring a patient's blood pressure configured to communicate with an implantable medical device for applying defibrillation shock pulses to the patient's heart.

The advantages and advantageous embodiments described above for the first aspect of the invention may also be applied to the method according to the second aspect of the invention, to the method according to the third aspect of the invention, to the medical device according to the fourth aspect of the invention and to the pressure sensor according to the fifth aspect of the invention such that it shall be referred to the above in this respect.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The idea of the invention shall subsequently be described in more detail with reference to the embodiments as shown in the drawings. Herein:

FIG. 1 shows a schematic drawing of a medical system in an implanted state in a patient and the programming device;

FIG. 2 shows an illustration of a medical system in communication with a programming device; and

FIG. 3 shows an illustration of a medical system operational on a patient's heart.

### DETAILED DESCRIPTION

Subsequently, embodiments of the invention shall be described in detail with reference to the drawings. In the drawings, like reference numerals designate like structural elements.

It is to be noted that the embodiments are not limiting for the invention, but merely represent illustrative examples.

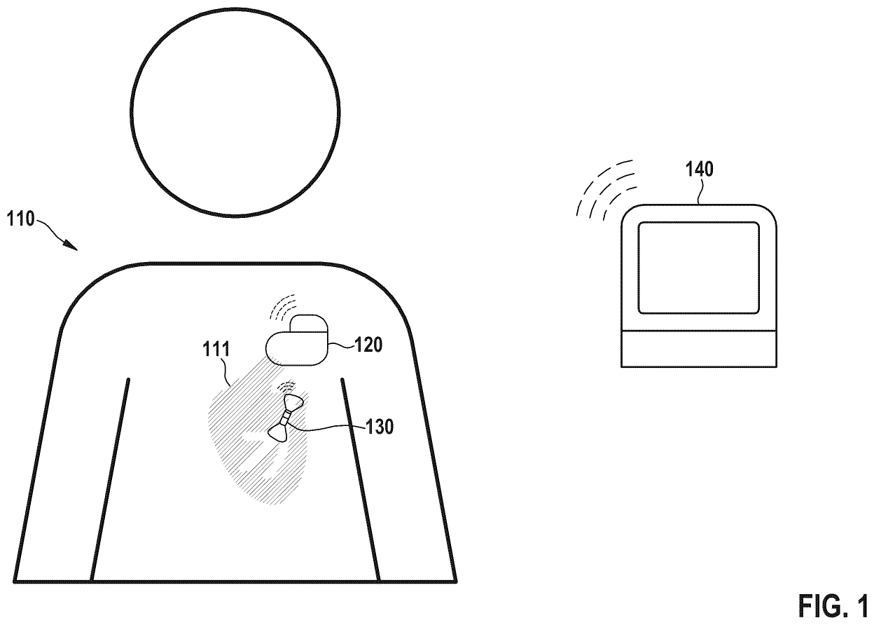

Referring to FIG. 1, a medical system is shown in an implanted state in a patient 110. The pressure sensor 130 is implanted at the patient's heart 111 in the pulmonary artery. It is operational to communicate wirelessly with a medical device 120 implanted near the patient's heart 111. The pressure sensor 130 measures the patient's blood pressure if it is in an operational state. A programming device 140 external to the patient 110 may be used by a physician to interrogate and/or program the medical device 120 and/or the pressure sensor 130 wirelessly.

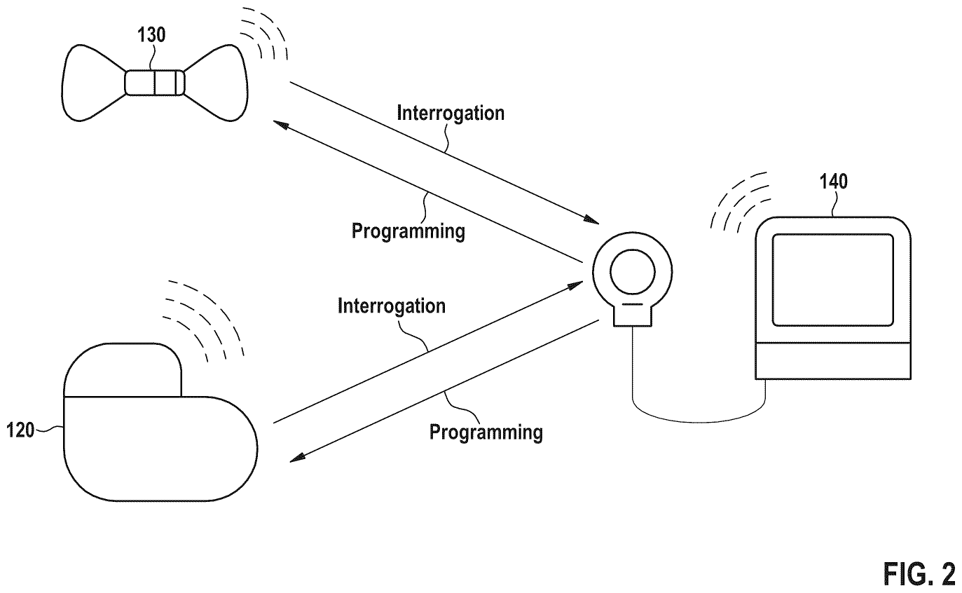

An interrogation and a programming process are shown schematically in FIG. 2. During the interrogation process, the programming device 140 may find the medical device 120 and the pressure sensor 130. The programming device 140 may then extract at least one parameter from the medical device 120 and the pressure sensor 130 in order to check whether the two implants are already informed of the presence of each other. During the programming process, the programming device 140 may set the at least one parameter so that the medical device 120 and pressure sensor 130 are informed of the presence of each other. If the medical device 120 knows about the presence of the pressure sensor 130, it can trigger the pressure sensor 130 if necessary. Triggering the pressure sensor 130 may, for example, be done by transmitting a wake-up signal to the pressure sensor 130. If the pressure sensor 130 knows about the presence of the medical device 120, it can get triggered by the medical device 120. Getting triggered by the medical device 120 may, for example, include waking up upon reception of a wake-up signal from the medical device 120.

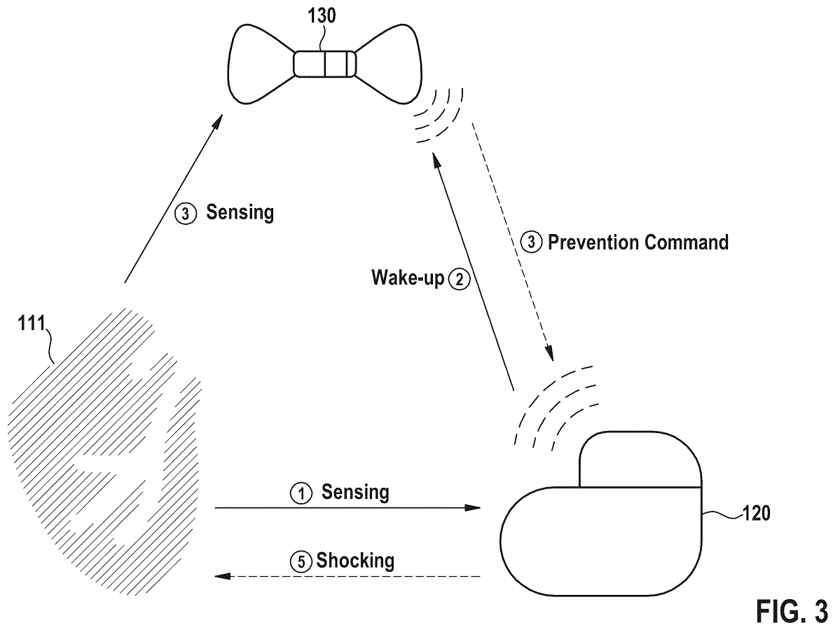

FIG. 3 shows an illustration of the medical system being operational on a patient's heart 111.

In a first step, the medical device 120 performs sensing 1 of an electrical signal of the heart 111. The medical device 120 may determine that a shock pulse should be applied if a cardiac disorder is detected upon analysis of the electrical signal. In a second step, the medical device 120 transmits a wake-up signal 2 to the pressure sensor 130 to wake up the pressure sensor 130 from a sleep state. The wake-up signal may be generated by the medical device 120 through a high-energy RF signal or a magnetic pulse. The wake-up signal triggers the pressure sensor 130 to switch from the sleep state to an operational state.

In a third step, the pressure sensor 130, in its operational state, detects the blood pressure by sensing 3 the heart 111. The sensing is done through a pressure measurement at the pulmonary artery. In a fourth step, the pressure sensor 130 transmits a prevention command 4 in the form of a shock prevention signal to the medical device 120. The shock prevention signal is transmitted if the pressure monitor detects cyclic variations of the patient's blood pressure during the pressure measurement (any of the information on the blood pressure such as the pulse may also be used here instead or in addition to its cyclic variations). If it does not detect cyclic variations of the blood pressure or does not detect cyclic variations within a predetermined range, e.g., a range with respect to duration and/or amplitude, the pressure monitor does not communicate with the medical device 120. The dependency of the transmission of the shock prevention signal on the detection result is indicated by a dashed line between the pressure sensor 130 and the medical device 120.

In a fifth step, the medical device 120 delivers a shock 5 to the patient's heart 111 if it did not receive the prevention command 4. During normal operations, this should be the case if the pressure monitor did not detect cyclic variations of the blood pressure (within the predetermined range). It also serves as a safeguard in case communication with the pressure sensor 130 has failed. The medical device 120 may only wait for a predetermined amount of waiting time of several seconds after transmission of the wake-up signal 2 before it delivers the shock. If the medical device 120 receives the prevention command 4, it will not deliver the shock. The dependency of the shocking 5 on the prevention command 4 is indicated by a dashed line between the medical device 120 and the heart 111.

The idea underlying the invention is not limited to the embodiments described above but may be implemented in an entirely different fashion.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### List of Reference Numerals

1 sensing by medical device
2 wake-up signal
3 sensing by pressure sensor
4 shock prevention signal
5 applying a shock pulse
110 patient
111 heart
120 medical device
130 pressure sensor
140 programming device

1. A medical system comprising an implantable medical device for applying defibrillation shock pulses to a patient's heart to treat an acute cardiac disorder,
and an implantable pressure sensor for measuring the patient's blood pressure, wherein the medical device and the pressure sensor are configured to communicate with each other, wherein the medical device is configured to decide whether a shock is applied after communicating with the pressure sensor.

2. (canceled)

3. The system of claim 1, medical device and the pressure sensor are configured to communicate with each other wirelessly, in particular, through intra-body communication.

4. The system of claim 1, wherein the medical device is configured to transmit a wake-up signal to the pressure sensor to wake up the pressure sensor from a sleep state so that the pressure sensor can measure the blood pressure.

5. The system of claim 4, wherein the wake-up signal comprises an RF signal and/or a magnetic pulse.

6. The system of claim 1, wherein the pressure sensor is further configured to transmit to the medical device information regarding the blood pressure and/or a shock prevention signal wherein the medical device is configured to decide whether or not to apply a shock based on the information and/or the shock prevention signal.

7. The system of claim 6, wherein the medical device is configured to wait for a predetermined amount of waiting time after sending the wake-up signal and to apply a shock if no communication from the pressure sensor is received after the waiting time has passed.

8. The system of claim 6, wherein the information comprises information on the presence or absence of cyclic variations of the blood pressure within a predetermined range and/or information on a detected blood pulse.

9. The system of claim 8, wherein the pressure sensor is configured to transmit the information on the detected blood pulse in the form of a plurality of individual signals, wherein each of the plurality of signals represents a single detected pulse beat.

10. The system of claim 9, wherein the medical device comprises a sensing arrangement for sensing an electrocardiogram signal, wherein the medical device is configured to compare the detected blood pulse with the electrocardiogram signal to cross-check the presence of heart beats.

11. A method for operating the system of comprising the steps of:
determining, by the medical device, that a shock is required;
transmitting, by the medical device, a wake-up signal to a pressure sensor to wake up the pressure sensor from a sleep state;
detecting, by the pressure sensor, the blood pressure;
transmitting, by the pressure sensor, to the medical device nothing or transmitting information regarding the blood pressure and/or a shock prevention signal; and
deciding, by the medical device, whether or not a shock is applied.

12. The method of claim 11, wherein deciding, by the medical device, whether or not a shock is applied comprises delaying application of the shock by a delay time if it decides not to apply a shock and determining, again, that a shock is required after the delay time has passed.

13. A method for setting up the system of claim 1 comprising the steps of:
providing a programming device configured to communicate with the medical device and the pressure sensor;
informing, by the programming device, the medical device the presence of the pressure sensor and the pressure sensor of the presence of the medical device; and
communicating, by the medical device, with the pressure sensor.

14. An implantable medical device for applying defibrillation shock pulses to a patient's heart configured to communicate with a pressure sensor for measuring the patient's blood pressure.

15. An implantable pressure sensor for measuring a patient's blood pressure configured to communicate with an implantable medical device for applying defibrillation shock pulses to the patient's heart.
